(19) United States
(12) Patent Application Publication
(10) Pub. No.: US 2025/0384984 A1
(43) Pub. Date: Dec. 18, 2025

(54) DYNAMIC ORDER PRESENTATION BASED ON SEQUENCING STATUS

(71) Applicant: Helix, Inc., San Mateo, CA (US)

(72) Inventors: John Anthony Pfeiffer, IV, Carmel Valley, CA (US); Jim Chih-Chun Chou, Cupertino, CA (US); Kirsten Mouritsen, San Francisco, CA (US); Mary von Kugelgen, Piedmont, CA (US)

(21) Appl. No.: 18/747,238

(22) Filed: Jun. 18, 2024

(51) Int. Cl. G16H 20/10; G16B 30/20; G16B 50/30; G16H 80/00

(52) U.S. Cl. CPC G16H 20/10; G16B 30/20; G16H 80/00; G16B 50/30

(57) ABSTRACT

Systems and methods herein provide for rapid patient information to healthcare providers such that the healthcare providers can make more informed decisions. One computer implemented method includes the use of a Clinical Decision Support (CDS) listener device. The CDS listener device detects, via a CDS hook, a request in a healthcare network from a healthcare provider for a patient. The CDS listener device forwards the request to a genomics server. The genomics server stores gene sequencing data of a plurality of genetic testing subjects. The genomics server then determines whether the patient is one of the plurality of genetic testing subjects, and, if the patient is one of the plurality of genetic testing subjects, the CDS listener device returns a sequencing status of the patient to the healthcare provider through the healthcare network.

### FIELD

The disclosure relates to the field of order processing related to genetic testing.

### BACKGROUND

Patients routinely undergo genetic testing to better understand the implications of certain genetic conditions that may impact their health. For example, when a patient is presented with a set of symptoms, those symptoms could be indicative of a genetic condition. As such, a healthcare provider may order a genetic test for that specific genetic condition. Genetic material is then acquired from a biological sample of the patient and shipped to a laboratory for testing in an environmentally controlled process. The laboratory may require days or even weeks to run the test before providing results.

And healthcare providers require robust and efficient tools to order tests in a timely manner. In particular, healthcare providers desire systems and methods that permit the rapid acquisition of laboratory tests, including sequencing results having clinical significance. Current methods of ordering are cumbersome in that they involve delays of weeks or longer before sequencing data can be retrieved, interpreted, and presented to the physician in a format that indicates clinical significance.

### SUMMARY

Embodiments described herein beneficially assist healthcare providers by providing rapid and valuable patient information to the healthcare providers such that the healthcare providers can make more informed diagnoses. For example, the embodiments herein may provide for dynamically suggesting relevant genetic tests relating to orders received in accordance with the Clinical Decision Support (CDS) hooks specification. The relevant genetic test recommendations are tailored based on whether or not the patient has already been sequenced by a laboratory performing the test.

In one embodiment, a computer implemented method includes detecting a request in a healthcare network from a healthcare provider for a patient. The request comprises a CDS hook. The method also includes forwarding the request to a genomics server. The genomics server is operable to store gene sequencing data of a plurality of genetic testing subjects. The method also includes determining whether the patient is one of the plurality of genetic testing subjects, and, if the patient is one of the plurality of genetic testing subjects, returning a sequencing status of the patient to the healthcare provider through the healthcare network. The detecting, forwarding, and returning may be implemented via a Session Initiation Protocol.

In some embodiments, the method also includes automatically generating a recommendation to provide genetic test data of the patient to the healthcare provider through the healthcare network in response to the detected request.

In some embodiments, the method also includes processing the CDS hook to identify a prescription from the healthcare provider for the patient, determining that the prescription is for a medicine where genetic testing is recommended, determining whether genetic test data of the patient exists with the genomics server, and, if the genetic test data of the patient exists with the genomics server, automatically generating a recommendation to provide the genetic test data of the patient to the healthcare provider through the healthcare network, else, if the genetic test data of the patient does not exist with the genomics server, automatically generating a recommendation for genetic testing of the patient to the healthcare provider through the healthcare network. In this regard, the method may also include processing a request from the healthcare provider through the healthcare network for genetic testing of the patient in response to the recommendation.

In some embodiments, the method also includes detecting another request in the healthcare network from the healthcare provider for another patient, the other request comprising another CDS hook, processing the other CDS hook to identify a prescription and a genetic test request from the healthcare provider for the other patient, automatically forwarding the genetic test request to a genetic testing laboratory to perform a genetic test on the other patient, and, when the genetic test is complete, storing data of the genetic test of the other patient with the genomics server, and forwarding the genetic test data of the other patient to the healthcare provider through the healthcare network. This may entail detecting that the healthcare provider has reviewed the genetic test data of the other patient, automatically forwarding the prescription to a pharmacy for electronic fulfillment of the prescription.

Other illustrative embodiments (e.g., systems and computer-readable media relating to the foregoing embodiments) may be described below. The features, functions, and advantages that have been discussed can be achieved independently in various embodiments or may be combined in yet other embodiments, further details of which can be seen with reference to the following description and drawings.

### DESCRIPTION OF THE DRAWINGS

Some embodiments of the present disclosure are now described, by way of example only, and with reference to the accompanying drawings. The same reference number represents the same element or the same type of element on all drawings.

FIG. 1 is a diagram depicting a sample processing architecture, in an illustrative embodiment.

FIG. 2 is a block diagram illustrating a genomics architecture, in an illustrative embodiment.

FIG. 3 is a data structure of a CDS hook, in an illustrative embodiment.

FIG. 4 is an interactive graphical user interface (GUI), in an illustrative embodiment.

FIG. 5 is a flowchart of a method for rapidly returning a sequencing status of a patient to a healthcare provider, in an illustrative embodiment.

FIG. 6 is a message diagram of a detected request from a healthcare provider for patient information, in an illustrative embodiment.

FIG. 7 is a message diagram of a detected prescription order from a healthcare provider and the return of a patient's sequencing status, in an illustrative embodiment.

FIG. 8 is a message diagram of a detected prescription order from a healthcare provider and the return of a patient's gene sequencing data, in an illustrative embodiment.

FIG. 9 is a message diagram of a detected prescription order from a healthcare provider and the return of pharmacogenomics (PGx) data to the healthcare provider, in an illustrative embodiment.

FIG. 10 depicts an illustrative cloud computing system operable to execute programmed instructions embodied on a computer readable medium.

### DESCRIPTION

The figures and the following description depict specific illustrative embodiments of the disclosure. It will thus be appreciated that those skilled in the art will be able to devise various arrangements that, although not explicitly described or shown herein, embody the principles of the disclosure and are included within the scope of the disclosure. Furthermore, any examples described herein are intended to aid in understanding the principles of the disclosure, and are to be construed as being without limitation to such specifically recited examples and conditions. As a result, the disclosure is not limited to the specific embodiments or examples described below, but by the claims and their equivalents.

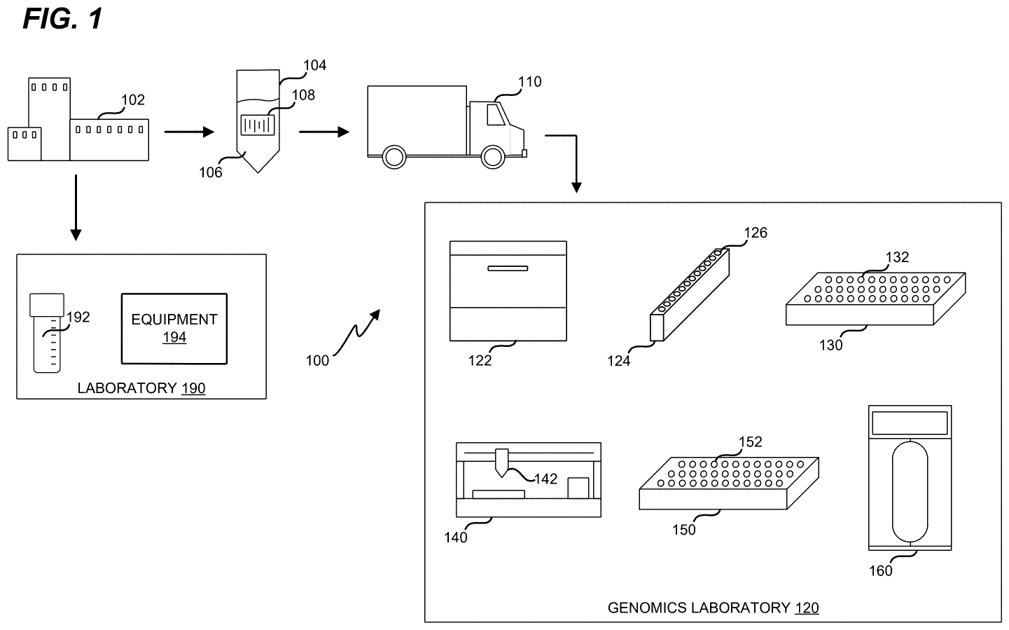

FIG. 1 is a diagram depicting a sample processing architecture 100 in an illustrative embodiment. Sample processing architecture 100 comprises any system or organizational structure for acquiring and sequencing biological samples in a high-volume, high-throughput manner. Sample processing architecture 100 may be utilized, for example, to collect and sequence genetic material (in the form of Ribonucleic Acid (RNA) or Deoxyribonucleic Acid (DNA)) found within thousands or tens of thousands of samples 106 daily, via multiple healthcare providers 102.

Healthcare provider 102 may comprise hospitals, clinics, practitioner offices, laboratories, surgical centers, etc. that engage in or facilitate the practice of medicine. In one embodiment, healthcare providers 102 comprise groups of hospitals that treat millions of patients. As a part of the practice of medicine, healthcare provider 102 acquires samples 106 for sequencing. For example, a healthcare provider 102 may acquire samples 106 as part of a population screening program, as part of medical treatment, etc. The specific amount of sequencing desired for a sample 106 may comprise a selected set of one or more genes, an exome, the entire genome of a patient, etc. The samples 106 are stored in sample containers 104, which may be accompanied by Customer Sample Identifiers (CSIs) 108. A delivery service 110 provides the samples 106 to a genomics laboratory 120 for processing.

Healthcare provider 102 may also acquire samples 192 for blood testing. These samples 192 may be provided to laboratory 190 for analysis via equipment 194 (e.g., a chemically treated test strip, biochemical assay, etc.), or may be analyzed by patients via at-home testing methods. Sample processing architecture 100 provides a technical benefit by allowing laboratory 190 and genomics laboratory 120 to specialize in different methods of analysis.

Procedures within genomics laboratory 120 related to genetics may include accessioning, sample plating, storage, extraction, library preparation, enrichment, and sequencing processes. These processes acquire genetic material from a sample 106, separate the genetic material from other constituents, duplicate the genetic material, and quantify the genetic material order to determine a swathe of sequence data, such as an exome or entire genome for a subject (e.g., a human patient, an organelle of a human patient, etc.). Although the procedures discussed herein are specific with regard to one method of sequencing, other techniques may be utilized in accordance with known standards in order to perform sequencing for samples 106. For example, although some of the techniques discussed herein relate to hybridization capture techniques, amplicon-based techniques may be used.

### Accessioning

Accessioning refers to receiving and preparing samples 106 for later laboratory processes. In one embodiment, accessioning includes receiving a batch of samples 106 (e.g., hundreds or thousands of samples 106) from one or more delivery services 110 each day for processing. For example, packages that each include tens or hundreds of samples 106 may be delivered to genomics laboratory 120 via the United States Postal Service (USPS), or a private package carrier.

Each sample 106 may be retained within a sample container 104, such as a five milliliter (mL) test tube. In this embodiment, the sample container 104 is sealed to prevent the sample 106 from being exposed to the environment and also to prevent the sample 106 from co-mingling with other samples 106. For example, the sample 106 may be sealed via a cap that is threaded, glued, press-fit, etc. At the time of delivery, the sample container 104 may further include a remnant of a sampling tool, such as a portion of a swab that was utilized to acquire the sample.

In many embodiments, a CSI 108 for the sample 106 is reported via a component affixed to or integrated with the sample container 104. The CSI 108 uniquely distinguishes the sample 106 from other samples 106 being received. For example, a CSI 108 may uniquely distinguish a sample 106 from other samples 106 in the same batch, other samples 106 received on the same date, other samples 106 received from the same healthcare provider 102, etc. A CSI 108 may be reported via a barcode label, Quick Response (QR) code label, Radio Frequency Identifier (RFID) chip, or any suitable visual, transmission-generating, or other physical component affixed to or integrated with the sample container 104.

In further embodiments, the sample container 104 is itself sealed within an external container such as a bag (not shown). Using an external container helps to prevent contamination, by ensuring that a technician at the genomics laboratory 120 does not contact biological material from the sample 106 that may exist on an outer surface of the sample container 104. Use of an external container may also be required by law (e.g., Department of Transportation (DOT) guidelines). Use of an external container additionally helps to prevent cross-contamination between samples 106. Furthermore, in embodiments where samples 106 may include blood or a pathogen, an external container provides an additional barrier to protect the health of technicians. The external container may additionally include documentation confirming the CSI 108, information for the subject that the sample was sourced from, and/or information indicating circumstances of sampling. The circumstances of sampling may include, for example, a sampling date, a sampling method, a location that the sample was acquired, a name or title for a person who performed the sampling, and/or additional notes.

In this embodiment, the sample 106 comprises a chemical solution. For example, the sample 106 may comprise a prepared aqueous solution such as a saline solution, or may comprise a bodily fluid such as blood, saliva, mucus, etc. In some embodiments each of the samples 106 fills between two and five milliliters of volume within its corresponding sample container 104.

The samples 106 further include genetic material such as Deoxyribonucleic Acid (DNA), Ribonucleic Acid (RNA), etc. In many instances, the genetic material is one of many constituent components within the sample 106. For example, the genetic material may exist within the nuclei of white blood cells that are included within the sample 106. In a further example, genetic material may exist within viruses or bacteria within the sample 106. In this embodiment, the genetic material is not yet isolated from the remaining constituent components of the sample 106.

After receipt of the samples 106, batches of the samples 106 (e.g., as stored within sample containers 104 and/or external containers) may be heated in ovens 122 to facilitate cell lysis. The temperature, and duration of heating, may be chosen such that pathogenic material within the samples 106 is rendered harmless, or such that cellular lysis occurs. For example, heating may occur at a temperature of between forty and eighty (e.g., fifty) degrees Celsius (C), for a period of time between fifteen and two hundred (e.g., thirty) minutes. In some embodiments, including embodiments wherein the samples 106 are primarily the contents of a blood draw, the heating step may be foregone.

Upon completion of heating, the batches of samples 106 are removed from the ovens 122. In one embodiment, sample containers 104 are removed from corresponding external containers, such as by cutting the external containers open. With the sample containers 104 now available for direct interaction, the sample containers 104 are inspected. As a part of this process, a technician or automated system may determine the CSI 108 for the sample 106, and may compare the CSI 108 to a CSI 108 listed on documentation provided in the external container. If there is a discrepancy between the CSI 108 on the sample container 104 and a CSI 108 listed in the documentation, the sample 106 may be flagged as having an error condition. Similarly, if the CSI 108 on the sample container 104 is damaged (e.g., abraded, heat-damaged, or water-damaged) and has become unreadable, the sample 106 may be flagged as having an error condition.

A technician or automated system may further inspect the contents of the sample container 104, via visual or other methods. If the sample 106 does not include an expected constituent component (or is otherwise non-compliant) then the sample 106 is flagged as having an error condition. For example, if the sample 106 is primarily saliva and includes a fluid that is not permitted (e.g., blood), includes an entire swab or no swab, appears to have a fractured or broken casing, or is outside of an expected range of volume (e.g., between two and five milliliters), then the sample 106 may be flagged as having an error condition.

Samples 106 that have not been flagged as having an error condition proceed to sample integration. In one embodiment, as a part of sample integration, the sample 106 is assigned a Laboratory Sample Identifier (LSI). The LSI uniquely identifies the sample 106 from other samples 106 received for the batch, received on the same day, processed in the same laboratory, and/or handled by the same organization performing sequencing. In many embodiments, the LSI is stored in a memory of a genomics server (e.g., within a laboratory sample database), and is uniquely associated with a corresponding CSI 108 for the sample. The LSI may also be associated with any error conditions reported for the sample 106.

In many embodiments, CSIs 108 originally provided with the samples 106 are in the form of a paper barcode. In such embodiments, the paper barcode may be printed in aqueous ink. This renders the barcode subject to degradation upon exposure to liquid in the laboratory environment, which is undesirable.

To ensure that each sample container 104 is capable of traveling through the genomics laboratory 120 without its identifier being physically degraded, a corresponding LSI may be indicated at the sample container 104. The LSI may be indicated via the application of a barcode label, Quick Response (QR) code, Radio Frequency Identifier (RFID) chip, or other visual, transmission-generating, or other physical component affixed to or integrated with the sample container.

In one embodiment, the LSI is printed onto a barcode label comprising rip-proof material (e.g., vinyl) in a water-insoluble ink. This implementation ensures that the barcode label is resistant to physical and chemical degradation. The barcode may be applied around an entire perimeter of the sample container 104, ensuring that the sample container 104 may be scanned from any angle.

In further embodiments, the element used to report the LSI is accompanied by a visually distinct mark that enables rapid confirmation by a technician that the sample 106 has been integrated into the laboratory environment. The visually distinct mark may comprise a colored ring (e.g., around an entire perimeter of the sample container), a logo, a physical feature, a stamp, etc.

### Sample Plating

With the samples 106 having been successfully integrated into the environment of the genomics laboratory 120 environment, the samples 106 are ready for analytics to be performed. To this end, the samples 106 are prepared for transfer to a sample microplate 130. The sample microplate 130 may be labeled with a unique identifier via similar techniques to those used for sample containers 104 above. The unique identifier distinguishes the sample microplate 130 from other sample microplates 130. In one embodiment, the sample microplate 130 comprises a solid body defining three hundred and eighty-four wells, distributed across sixteen rows and twenty-four columns, each well having a capacity of between thirty and one hundred microliters. In a further embodiment, the sample microplate 130 comprises a solid body defining ninety-six wells, distributed across eight rows and twelve columns, each well having a capacity of between one hundred and three hundred microliters. Any suitable number and arrangement of wells may be selected as a matter of design choice.

As a part of preparing the samples 106 for transfer to the sample microplate 130, a technician may place sample containers 104 onto a rack 124, and scan each sample container 104 to determine an LSI for each location 126 (e.g., each container receptacle) on the rack 124. In some embodiments, the rack 124 is assigned a unique identifier that distinguishes it from other racks 124. The rack 124 may be labeled with a unique identifier using techniques similar to those used for sample containers 104. The technician, or automated machinery such as a server operating an optical scanner, may then associate the unique identifier for the rack 124, along with the locations 126 assigned to the samples 106, with the corresponding LSIs of the samples 106 stored at the rack 124.

The technician additionally unseals the sample containers 104. Unsealing of sample containers 104 may be a deeply labor-intensive process, particularly when laboratory processes are performed at scale to handle tens of thousands of samples 106 per day. Thus, a technician may utilize automated tooling to enhance the speed at which sample containers 104 are unsealed. The tooling may, for example, unscrew, cut, or drill each sample container 104, in order to make the sample 106 within available for physical transfer to the sample microplate 130.

One or more racks 124 of samples 106 are provided to a Liquid Handler (LH) 140, such as an automated robot that operates an end effector 142 in accordance with one or more Numerical Control (NC) programs to transfer liquids between wells via arrays of micropipettes. An LH 140 is also known as a “Liquid Handling System.” LH 140 may comprise, for example, a Hamilton Microlab Star Liquid Handling System.

In this embodiment, the LH 140 proceeds to transfer a portion of each sample 106 at a rack 124 to a well 132 within the sample microplate 130 that is not shared with other samples 106. For example, the well 132 for each sample 106 may be predetermined in accordance with a control program used by the genomics laboratory 120. In one embodiment, the LH 140 transfers the portions of the samples 106 to the wells 132 of the sample microplate 130 by providing instructions to actuators, piezoelectric elements, and/or pressure systems operating the end effector 142. In such an embodiment, the end effector 142 may align its array of micropipettes with the sample containers 104 to retrieve portions of the samples 106. Furthermore, in such an embodiment, the end effector 142 may dynamically align its array of micropipettes with the sample microplate 130 to deposit the portions of the samples 106 at the wells 132.

Because there is a known relationship between locations 126 at the rack 124 and wells 132 of the sample microplate 130 (e.g., as indicated by row and column), contents of the memory of a genomics server (e.g., a laboratory sample database) may be updated to indicate the well 132 storing genetic material for each sample 106. In one embodiment, the memory is further updated to associate a unique identifier for the sample microplate 130 with the samples 106 stored therein.

In one embodiment, programmed instructions for the LH 140 may direct the end effector 142 to position itself above a set of disposable tips, descend into the tips to attach the tips, reposition the end effector 142 above the rack of sample containers 104, adjust spacing between micropipettes within the array, descend until the tips reach the sample containers 104, draw liquid from the sample containers 104, deposit the liquid into a well at the sample microplate 130, and then dispose of the tips. Such a process may be repeated across sample containers 104 stored on multiple racks until the sample microplate 130 is filled with portions from the samples 106. In one embodiment, one or more wells 132 on the sample microplate 130 are filled with a control reagent instead of a portion of a sample 106.

The amount of liquid drawn from each sample container 104 may comprise a small fraction of the overall volume of the sample container 104. For example, an amount of liquid drawn may comprise several microliters, such as between two and ten microliters. Upon completion of transfer from the sample containers 104 to the wells, the sample microplate 130 may be covered with a liquid and/or gas-impermeable layer, such as foil or paraffin. Sample containers 104 remaining on the racks may be resealed, for example with pressure-fit caps having a color distinct from an original color for the sample containers. With accessioning now complete for the sample microplate 130, the sample microplate 130 is transferred to a next section of the laboratory for processing.

### Storage

In one embodiment, accessioned samples 106, samples 106 ready for analytics, and/or samples 106 that have already been sequenced, are stored for later use. For example, samples 106, sample containers 104, and/or sample microplates 130 may be stored at room temperature, or may be cryogenically frozen at a low temperature (e.g., negative eighty degrees Celsius) and arranged in racks for later retrieval. Samples 106 may be preserved for periods of days or years, enabling rapid re-testing to be performed for subjects without the need for re-acquiring genetic material. Storage of the samples 106 provides notable value in the event that contents of a well 132 used for sequencing do not meet with rigorous quality control standards. Specifically, storage enables re-sampling to occur in the event that there is a desire to resequence a sample 106.

### Extraction

Sample microplates 130 are transferred to a portion of the genomics laboratory 120 dedicated to extraction of the genetic material. The segment of the laboratory 120 that performs extraction and other pre-amplification operations may be sealed from, and/or positively pressurized relative to, other portions of the genomics laboratory 120.

During extraction, a sample microplate 130 is acquired and provided to an LH 140. The LH 140 that performs extraction may be different from the LH 140 that performs sample plating. The LH 140 may apply a reagent to each well 132 that lyses cells within each well. For example, this may be performed in order to lyse white blood cells containing genetic material for a human, or may comprise lysing other types of cells to expose other types of genetic material. The reagents used for pre-amplification processes may be stored at the LH 140 in a temperature-controlled manner, and may even be vibrated or mixed on a regular basis to ensure that the reagents are evenly distributed in suspension.

In one embodiment, extraction further includes an LH 140 aspirating and dispensing reagents that selectively bind to genetic material released from the lysed cells. This process may include applying a bead (not shown) to the well 132. In one embodiment, the beads comprise magnetic beads that selectively bind to the genetic material (e.g., DNA). This allows for isolation and purification of the genetic material while contaminants remain in solution. In one embodiment, the magnetic bead is drawn to a magnetic base at or under the sample microplate 130. After the genetic material has been drawn to the bead, and after the bead has been secured to the base of the well, a flushing step may be performed wherein remaining fluid in each well is washed away. This ensures that potential impurities are removed from the well. The LH 140 may further add or remove fluid from each well 132 to perform additional concentration and/or elution of the genetic material, and may transfer fluid from the wells 132 of the sample microplate 130 to wells 152 of a genome stock microplate 150. The genome stock microplate 150 may be labeled with a unique identifier, and the contents of each well 152 of the genome stock microplate 150 may be associated with a corresponding LSI. In all phases of operation, the LH 140 is operated to ensure that fluid is not transferred between wells 152, as this results in contamination.

In one embodiment, a portion of fluid is removed from each well 152 of the genome stock microplate 150 for quality control purposes. Concentration of genetic material within the wells 152 may be confirmed via testing of this fluid, such as by application of a dye that reacts with the genetic material at known levels of fluorescence for known concentrations.

### Library Preparation

After extraction is completed, library preparation may be performed for the contents of the genome stock microplate 150. The bead for each well, including ionically bonded genetic material, is transferred to a distinct well of a library preparation microplate (not shown). The library preparation microplate includes an identifier that uniquely distinguishes it from other library preparation microplates, and the LSI associated with each well on the genome stock microplate 150 may be mapped to a corresponding well on the library preparation microplate.

The library preparation microplate may be transferred to a new portion of the genomics laboratory 120 that is sealed from, and/or positively pressurized relative to, other portions of the genomics laboratory 120 that do not perform amplification of genetic material. This feature helps to prevent amplified genetic material from entering portions of the laboratory where genetic material has not been amplified, which could result in contamination. The transfer process may be performed by placing a library preparation microplate into an airlock at the pre-amplification portion of the genomics laboratory 120, sealing the airlock, and then retrieving the library preparation microplate from the airlock via the amplification portion of the genomics laboratory 120.

In one embodiment, a reagent is applied to each well of the library preparation microplate. The reagent ionically bonds to the surface of the bead within the well, and does so more strongly than the genetic material. This releases the genetic material from the surface of the bead of each well, enabling the genetic material to be chemically interacted with.

Library preparation may include normalization of a concentration of genetic material in each well of the library preparation microplate. Library preparation further includes fragmentation of the genetic material via an enzyme or via the application of physical forces. During this process, the entire genome (e.g., roughly three billion base pairs for a human genome), may be fragmented into pieces. In one embodiment, the pieces vary between three hundred and four hundred base pairs in length. These pieces are known as nucleic acid fragments.

In this embodiment, the nucleic acid fragments undergo adaptor ligation and indexing in accordance with known techniques. For example, this may comprise Next Generation Sequencing (NGS) library preparation processes defined by Illumina. Next, a limited amount of Polymerase Chain Reaction (PCR) amplification is performed upon the library. The resulting solution is then purified and eluted via operation of an LH 140.

During library preparation, one or more reference samples of genetic material, distinct from the genetic material found in the samples, may be added to wells of the library preparation microplate. The reference samples do not include genetic material received from a customer, but rather include known sequences of base pairs. The reference samples serve as controls to ensure that processes are carried out with sufficient quality.

Upon completion of library preparation, desired fragments of the genetic material (e.g., thousands or millions of distinct fragments of the genetic material, each corresponding with a different portion of a genome of the subject) have been ligated to predefined adapters (e.g., DNA adapters) that bind with the genetic material. Each of the adaptor-ligated fragments is referred to as a “library.”

In further embodiments, the probes applied to each well of the library preparation plate include chemical identifiers (colloquially referred to as “barcodes”) that are distinct from each other. The use of a different chemical identifier for probes applied to each well of the library preparation microplate enables sequencing to later be performed for multiple subjects on the same flow cell, without conflating sequencing results for those subjects.

The library preparation process may further comprise controlling a concentration of the genetic material in each well, and purification and/or elution of the resulting material. Similar to the processes performed after extraction of genetic material, concentration of genetic material after library preparation may be confirmed for each well via testing.

### Enrichment

After library preparation, enrichment processes may be performed in order to either directly amplify (e.g., via amplicon or multiplexed PCR) or capture (e.g., via hybrid capture) predefined libraries. This enhances the ease of sequencing desired portions of the genome.

In one embodiment, during enrichment, customized biotinylated oligonucleotide probes are applied to the libraries. The probes selectively hybridize genetic material occupying desired portions of the genome for the genetic material, such as specific genes, or the entire exome. Magnetic beads bind to biotin molecules in the probes to attach the hybridized material to the magnetic beads. Magnetic forces capture the beads in place, enabling remaining fluid within each well to be removed or washed out, thereby removing impurities, and leaving only the genetic material that is desired. Genetic material may be released from the beads in a similar manner to that discussed above for prior processes.

In a further embodiment, hybrid capture target enrichment is performed. During this process, the probes comprise tailored oligonucleotides that are chosen to bind to the genetic material. The range of probes may be tailored as a group to bind to specific alleles, specific genes, the exome, the entire genome, etc. That is, each probe may bind to a nucleic acid fragment at a specific location on the genome, and the range of probes may be selected to ensure that alleles, genes, the exome, or the entire genome of the subject being considered is acquired. Utilizing probes in this manner may enhance efficiency of the sequencing process, by foregoing the need to sequence all of the roughly three billion base pairs found in the human genome.

The enrichment process may further comprise controlling a concentration of the genetic material in each well, and purification and/or elution of the resulting material. Similar to the processes performed after extraction of genetic material, concentration of genetic material after enrichment may be confirmed for each well via testing.

### Sequencing

Sequencing may be performed according to any of a variety of techniques, including short-read and long-read techniques, via sequencing equipment 160 (e.g., an Illumina NovaSeq X sequencing machine). In one embodiment, the sequencing is performed as Sequencing by Synthesis (SBS). For example, sets of enriched libraries of genetic material bound to probes in earlier steps may be transferred to a flow cell, and annealed to oligonucleotide probes within the flow cell. At this stage, the contents of multiple wells may be applied to the same flow cell, because the libraries within those wells are tagged with the chemical identifiers referred to above. In one embodiment, the chemical identifiers comprise nucleotide sequences that are detectable during the sequencing process to determine a corresponding LSI.

Complementary sequences may then be created via enzymatic extension to create a double-stranded portion of genetic material. The double-stranded genetic material may then be denatured, and the library fragment may be washed away. Bridge amplification may then be performed to create copies of the remaining molecule in a localized cluster. For example, a cluster may comprise twenty to fifty copies of the same molecule, localized to a location the size smaller than a pinhead on the flow cell.

In this embodiment, sequencing primers are annealed to library adapters in order to prepare the flow cell for SBS. During SBS, the sequencing primer uses reverse terminator fluorescent oligonucleotides, one base per cycle, for a number of cycles (e.g., one hundred and fifty cycles) in the forward direction. After the addition of each nucleotide, clusters are excited by a light source, resulting in fluorescence which can be measured. The emission wavelength and signal intensity for each cluster determines a base call for that cluster. Fluorescent moieties are then flushed from the flow cell. A chemical group blocking a 3′ end of the fragment is then removed, enabling a subsequent nucleotide to be read. This tightly controls nucleotide addition and detection.

Additionally in this embodiment, base calls across cycles at the same physical location on the flow cell occur at the same cluster, and hence indicate sequential reads for copies of the same fragment of the genetic material. After each cycle, denaturing and annealing are performed to extend the index primer. A complementary reverse strand is created and extended via bridge amplification. The reverse strand is then read in the reverse direction for a number of cycles, in a manner similar to reads in the forward direction.

Depending on whether a complete human genome, or another set of genomic data, is being tested, different reagents (e.g., probes, primers, etc.) may be chosen. That is, different reagents may be utilized for library preparation for a pathogen (e.g., bacteria, virus) or an organelle (e.g., mitochondria) than for a human genome. Pathogens exhibiting Ribonucleic Acid (RNA) genomes may have their genetic material translated to DNA before sequencing, enrichment, and/or library preparation are performed, via known techniques, such as Next Generation Sequencing (NGS) techniques.

Throughout the processes discussed above, the laboratory environment may be carefully controlled to ensure quality. For example, temperature within each segment of the laboratory may be carefully monitored and controlled, and ultraviolet lighting or other features capable of inactivating genetic material may be carefully positioned to ensure that contamination does not occur.

### Bioinformatics

Sequencing data may be stored in any suitable format. In one embodiment, raw sequencing data generated during synthesis is stored in a file format such as Binary Base Call (BCL). This raw data may be fed to an analytical pipeline such as a cloud-based computing environment. Raw sequencing data may be processed by the pipeline into a second format, such as a text-based FASTQ format, that reports quality scores. The second format may then be analyzed to perform alignment of sequence reads to a reference genome, such as a reference genome reported in a Browser Extensible Data (BED) file. The aligned sequence data may be reported as a Binary Alignment Map (BAM) file or Compressed Reference-oriented Alignment Map (CRAM) file. The aligned sequence data may then be called, resulting in a Variant Call Format (VCF) file reporting called variants at each location of the genome that was sequenced, together with secondary metrics such as quality indicator metrics. As used herein, a variant comprises a unique combination of genetic information, in the form of consecutive base pairs at a specific set of locations (e.g., genomic coordinates) along a portion of a chromosome. Each variant is distinguished from other variants by having a different combination of base pairs along the set of locations. This may be due to Single Nucleotide Polymorphisms (SNPs) which relate to common single nucleotide changes, Single Nucleotide Variants (SNVs) which relate to rare nucleotide changes, insertions and/or deletions (Indels) which relate for example to the insertion or deletion of less than thirty base pairs, or differing numbers of repetitions, Copy Number Variants (CNVs), which relate to larger insertions or deletions, translocations, inversions, other types of genetic variants, or even combinations of variants, such as haplotypes or Multi-nucleotide variants (MNVs).

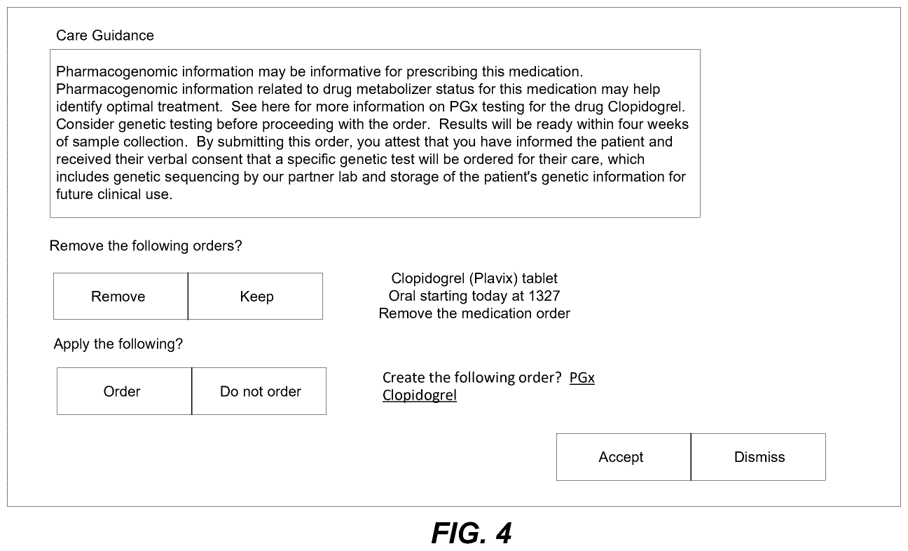

The called sequence data may be provided to a data analyst via a User Interface (UI), such as a Graphical User Interface (GUI) presented via a display. An example of such is illustrated in FIG. 4. The technician may then validate the resulting variants called from the sequence data and release it for reporting to subjects, healthcare providers, and/or scientists.

### Genomics Architecture

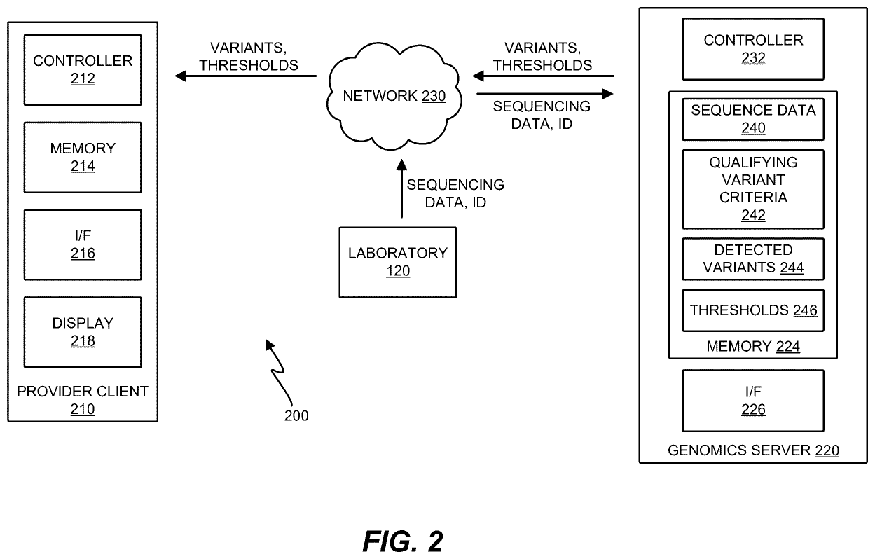

FIG. 2 is a block diagram illustrating a genomics architecture 200 in an illustrative embodiment. Genomics architecture 200 comprises any combination of systems and devices operable to review, process, and/or control access to sequencing data, including sequencing data received from genomics laboratory 120. In this embodiment, genomics architecture 200 comprises a genomics server 220 which receives sequencing data and identifiers (e.g., CSIs 108, LSIs, etc.) from genomics laboratory 120, via network 230.

Genomics server 220 receives the sequencing data via interface (I/F) 226, such as an Ethernet interface, wireless interface compliant with Institute of Electrical and Electronics Engineers (IEEE) 802.11 standards, or other physical interface capable of transmitting and receiving digital data. The sequencing data 240 is stored in memory 224 for the population of patients (e.g., millions of patients) that have been sequenced by laboratory 120, and may be maintained in any suitable format. Examples of such formats include CRAM, VCF, BAM, and others. Memory 224 may store, for example, sequence data 240 describing multiple patients, and this sequence data 240 may be maintained in a de-identified format to facilitate the advancement of research. Memory 224 may be implemented via a cloud storage service, or may comprise a storage medium such as a hard disk or flash memory device.

Memory 224 additionally stores qualifying variant criteria 242, detected variants 244, and thresholds 246 for diagnosis and/or treatment of various conditions associated with the variance 244. Examples include Centers for Disease Control (CDC) Tier 1 conditions, cardiomyopathy, pharmacogenomics sensitivities, BRCA1 and BRCA2 gene variants associated with breast cancer, GCK variants associated with type 2 diabetes, etc. In one embodiment, the portion of memory 224 storing these components is distinct from the portion of memory 224 storing sequence data 240.

Controller 232 manages the operations of genomics server 220, and may for example analyze sequence data 240 to identify detected variants 244, control access and authentication related to sequence data 240, communicate with one or more provider clients 210, and/or perform additional operations. Controller 232 may be implemented, for example, as custom circuitry, as a hardware processor executing programmed instructions, as a combination of shared hardware processing resources implementing a compute service, or some combination thereof.

Genomics architecture 200 further comprises provider client 210, which is configured to receive information regarding detected variants 244 and/or thresholds 246. In this embodiment, provider client 210 includes a controller 212, a memory 214, an interface (I/F) 216, and a display 218. Controller 212 manages the operations of the provider client 210, and may be implemented, for example, as custom circuitry, as a hardware processor executing programmed instructions, or some combination thereof. Memory 214 comprises information for interpreting the data received via I/F 216. Display 218 may comprise a projector, screen, etc. for presenting information to a user of provider client 210.

### Interpreting Sequencing Data

After sequencing data for the patient has been acquired (e.g., as an accompaniment to blood testing, in a prior event that provided a sample 106, etc.), sequencing data for the genes is reviewed for that patient by controller 232 of genomics server 220. For example, the sequencing data may be reviewed across the entire genome or exome, including for one or more genes that contribute to a specific phenotype or disease.

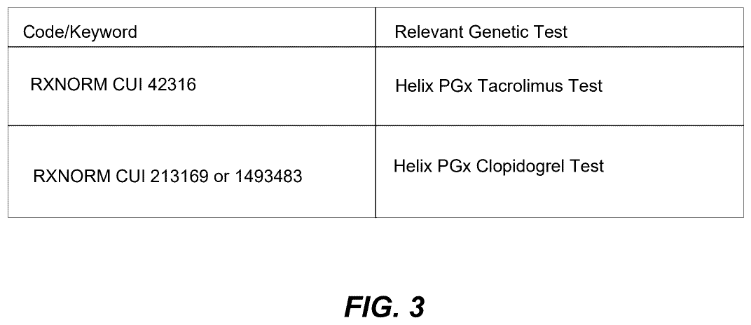

With the foregoing description provided of illustrative systems for sample intake and sequencing, the following FIGS. 3-10 recite illustrative techniques for utilizing sequencing data (e.g., acquired using the systems of FIGS. 1-2) to facilitate diagnostic decisions.

As discussed above, a sample of genetic material for a patient may be received (e.g., from a healthcare provider) and sequenced (e.g., via an assay at a laboratory). Multiple analytical tools may be used to analyze the genomic data for the patient in order to determine results for one or more tests (e.g., diagnostic tests, population screening tests, etc.). As used herein, an analytical tool comprises a computer-implemented program, function, or code that analyzes genetic data to generate a quantitative or qualitative or result. As used herein, a diagnostic test comprises a request for genomic data

The initial sequencing process performed at the laboratory may acquire a substantial amount of genetic data. This amount of genetic data does not need to correspond with the scope of an initial test that caused the sample to be sent to the laboratory. For example, even though the initial test may only consider a small portion of a gene, the laboratory may sequence an entire gene, the entire exome, the entire exome and selected additional regions, or the entire genome of the patient.

Generally, the analytical tools used are software programs that perform bioinformatic operations, such as sequence alignment, variant calling, haplotype calling, and/or imputation for genetic data. Other analytical tools may be used for calling ancestry of the patient. One example of a tool that may be used includes a Burrows-Wheeler Aligner (BWA) process to map low-divergent sequences (e.g., in a FASTQ format generated by a sequencing machine) against a large reference genome, such as a human genome reported in a Binary Alignment Map (BAM) file. Another example of a tool that may be used includes the Genome Analysis Toolkit (GATK) from the Broad Institute in order to perform variant calling.

In any case, a listener service (e.g., CDS listener 610 described below) receives Clinical Decision Support (CDS) hooks, which are generated by one or more healthcare provider networks. CDS hooks are defined by the CDS hooks specification, which is one of the Health Level 7 (HL7) standards. Each healthcare provider may be operated by a separate third party and may independently select specific types of CDS hooks the listener service detects. For example, a healthcare provider network may provide CDS hooks that include draft orders for pharmaceutical prescriptions, CDS hooks that refer to cardiac tests such as Electrocardiograms (ECGs), CDS hooks for chest imaging, etc.

Examples of a CDS hook are shown in FIG. 3. In this embodiment, the CDS hook RXNORM CUI includes an identifier 42316 that is used to prescribe PGx testing for the drug Tacrolimus. The CDS hook RXNORM CUI having an identifier 213169 or 1493483 is used to prescribe PGX testing for the drug Clopidogrel. The CDS hook may also include a patient identifier (e.g., a medical record number, or “MRN”) that links patient identifiers in the genomics server 220 to patient identifiers in the healthcare provider network.

The listener service reports to the genomics server 220. The genomics server 220 includes records that identify patients which have been sequenced at the genomics laboratory 120, and attempts to link patient identifier information within CDS hooks to the records of patients that have been sequenced (e.g., by finding patients with identifiers at the genomics server 220 that match identifiers found in CDS hooks). The genomics server 220 is capable of accessing one or more analytical tools for analysis of gene sequencing data for patients. The sequencing data may comprise raw sequencing data, such as FASTQ sequencing data, or formatted sequencing data, such as a BAM file or Variant Call Format (VCF) data.

The genomics server 220 stores the data and maps specific clinical indications to the data, which are represented in the CDS hook's context data as discrete codes (e.g., International Classification of Diseases (ICD) codes, Medical Prescription Normalized (RxNORM), Current Procedural Terminology (CPT) codes, etc.) and/or phrases to specific relevant genetic tests. The genomics server 220 further stores data that maps specific relevant genetic tests to analytical tools. Each relevant genetic test is associated with an analytical tool that performs analysis of sequencing data. In some embodiments, multiple genetic tests are associated with a single analytical tool (i.e., a specialized program for interpreting genetic data).

During operation, healthcare providers generate CDS hooks as a part of managing the health of their patients. This may be performed, for example, by placing an order within an Electronic Health Record (EHR), preparing a draft order within an EHR, etc. The generated CDS hooks are reviewed (e.g., by the healthcare provider network or the listener service) for specific criteria. The criteria may relate to the type of CDS hook and/or the content of the CDS hook. For example, the healthcare provider network may identify “order select events”, “patient view events”, etc., and/or may review the context portions of those events for specific keywords, such as medication names or relevant codes. CDS hooks that meet the specific criteria for their healthcare provider network are hereinafter referred to as “qualifying hooks,” and are provided to the listener service.

In response to detecting a qualifying hook, the listener service provides the qualifying hook to the genomics server 220. The genomics server 220 reviews the contents of the qualifying hook (e.g., as stored within the context portion, the draft orders portion, and/or the prefetch portion of the qualifying hook). The genomics server 220 reviews the contents for keywords that are associated in memory with relevant genetic tests. For example, prefetch data referring to cardiovascular or cardiomyopathy conditions may be associated with a relevant genetic test for cardiomyopathy. If no relevant genetic tests have been detected, further processing of the CDS hook by the genomics server 220 may cease, resulting in the listener service returning a 200 (OK) response (e.g., a Session Initiation Protocol) to complete the workflow without requiring input from the provider.

Next, if one or more relevant genetic tests have been detected, the genomics server 220 determines whether sequencing data (e.g., whole exome data, whole genome data, etc.) for the patient referenced in the qualifying hook is already accessible. This may be determined by searching for sequencing records or statuses that are linked with a patient identifier provided for the CDS hook. If the patient has already been sequenced by a laboratory (e.g., the genomics laboratory 120 of FIG. 1) reporting to the genomics server 220, then the genomics server 220 may generate an expedited genetic testing recommendation. In one embodiment, to enable this feature to operate in a real-time manner (i.e., to provide real-time feedback to healthcare providers on what type of order to initiate), a cloud compute node (e.g., e.g., lambda provided by Amazon) operates as a real-time cache to return sequencing status. In order to ensure that results can be provided in real-time (e.g., within less than several seconds), lambda warmers may be utilized. For example, open source libraries may be provided to “warm” Lambda functions via a pinging mechanism. This approach uses EventBridge rules to schedule invocations of the function every minute to help keep the execution environment active. As a result, this can increase the likelihood of using a warm environment when a function is invoked. Thus, lambda warmers increase the likelihood of using a warm function when invoking the lambda. As an additional step to enhance responsiveness, provisioned concurrency may be implemented in order to have initialization activities for the lambda occur ahead of lambda invocation.

The expedited genetic testing recommendation refers to the qualifying hook, and indicates the availability of expedited genetic testing results having a fast Turn Around Time (TAT), such as within several minutes, an hour, or a day. The expedited genetic testing recommendation includes CDS context information as part of a qualifying hook response, including any draft or unsigned orders. This recommendation is part of the CDS hooks order selection workflow process, and is provided as a suggested action within the CDS Response. Healthcare providers may then choose to order the suggested test, which contains the expedited workflow instructions (e.g., such as bypassing sample collection and including the expected TAT). Incremental to a regular order, analytics around CDS presentation and selection may be recorded in order to analyze conversion rates. Once chosen, the suggested test is transmitted to the laboratory as an HL7 message. In the case of PGx testing that impacts prescribing recommendations, the healthcare provider may postpone finalizing the original order until after expedited genetic testing results have been received.

Alternatively, if the patient has not already been sequenced, then the genomics server 220 generates a standard genetic testing recommendation. The standard genetic testing recommendation refers to the qualifying hook, and indicates the availability of standard genetic testing results having a TAT that is longer than expedited genetic testing (e.g., several days or weeks). The standard genetic testing recommendation is provided as a suggested action within the CDS Response. Healthcare providers may then choose to order the suggested test, which contains the standard workflow instructions (e.g., sample collection including the expected TAT), which is then transmitted to the genomics laboratory 120 as an HL7 message. The HL7 message is an ORM message which contains order information to sequence the patient, alongside physical sample information, such as barcode and collection time. ORM messages are order entry messages that may be used to facilitate orders and results-based workflows. In an embodiment for pharmacogenomics, an accompanying request may be used to perform both wet lab sequence orders and pharmacogenomics orders, for example.

The healthcare provider may postpone finalizing the original order until after standard genetic testing results have been received. Standard genetic testing may include physically acquiring a sample of genetic material from the patient, sending the sample to the genomics laboratory 120 for processing, and then sequencing the sample. This may be followed by utilizing an analytical tool for the genetic test to process resulting sequencing data.

To illustrate, a healthcare provider interacts with an ordering system within the EHR, which functions as a CDS Client. The healthcare provider may order a prescription for clopidogrel for a first patient. The healthcare provider network identifies the corresponding CDS hook for the order (e.g., an “order select event”) as referring to clopidogrel, determines that the CDS hook is a qualifying hook based on its reference to clopidogrel, and sends the CDS hook to a CDS listener Service. And the listener service provides the CDS hook to the genomics server 220.

The genomics server 220 determines whether it already stores whole exome sequencing data for the first patient (e.g., based on a patient identifier provided with the CDS hook). The genomics server 220, in response to determining that the whole exome sequencing data is already stored for the first patient, then generates a recommendation for expedited PGx testing for the first patient, prior to finalizing the order for the prescription. The TAT for this testing is reported as between five and ten minutes. The recommendation is provided as a CDS Response containing suggested actions. The EHR receives the recommendation, and generates an interactive Graphical User Interface (GUI) element to order the PGx testing before prescribing clopidogrel. The interactive GUI element displays the TAT for this testing as well. Based on this, the healthcare provider may immediately order the expedited PGx testing before ordering the prescription.

In another example, the healthcare provider may order a prescription for clopidogrel for a second patient, which is processed in a similar manner to the order for the first patient, until the genomics server 220 determines that no sequencing data is available for the second patient. The genomics server 220 then generates a recommendation for standard PGx testing for the second patient, prior to finalizing the order for the prescription. The TAT for this testing is reported as between one and three weeks. The recommendation is provided as a CDS Response containing suggested actions. The EHR receives the recommendation, and generates an interactive GUI element to order the PGx testing before prescribing clopidogrel. The interactive GUI element displays the TAT for this testing as well. Based on this, the healthcare provider may immediately order the standard PGx testing prior to ordering the prescription.

If the healthcare provider signs the order, then a request is sent to the genomics server 220 to initiate sampling and sequencing for the second patient. This may result in the genomics server 220 identifying contact information for the second patient, and providing a link for the second patient to provide consent via a portal. This initiates a chain of events for acquiring a sample of genetic material from the second patient, and then performing the requested genetic test after the patient has been sampled. The sequencing data may then be acquired and processed by a suitable analytical tool. Corresponding PGx test results are then reported to the healthcare provider within the EHR as an HL7 Result Message. An example of the HL7 Result Message is illustrated in FIG. 4.

The various embodiments herein are described with reference to sample processing architecture 100 of FIG. 1 and genomics architecture 200 of FIG. 2, but those skilled in the art will appreciate that these embodiments may be performed in other systems. The steps of flowcharts described herein are not all inclusive and may include other steps not shown. The steps described herein may also be performed in an alternative order.

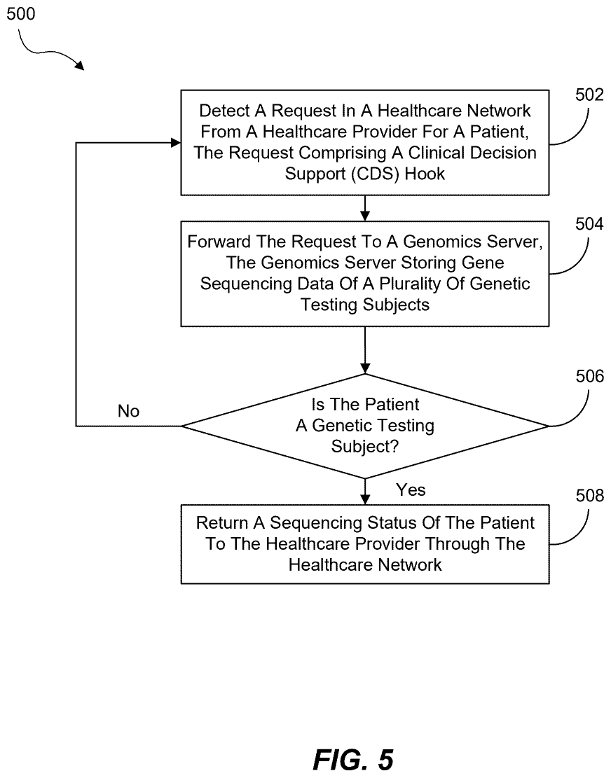

With this in mind, FIG. 5 is a flowchart of a method 500 for processing a request from a healthcare provider in a healthcare network. In this embodiment, a CDS listener device is communicatively coupled to a healthcare network server and is operable to detect a request in the healthcare network from a healthcare provider for a patient, in the process element 502. The request comprises a CDS hook. Once the CDS listener device identifies the CDS hook, the listener device forwards the CDS hook to a genomics server, such as the genomics server 220 of FIG. 1, that stores gene sequencing data of a plurality of genetic testing subjects, in the process element 504. The genomics server then determines whether the patient is one of the genetic testing subjects whose gene sequencing data is stored therewith, in the process element 506. If so, the genomics server returns a sequencing status (e.g., “sequenced,” or “not sequenced”) of the patient to the listener device which, in turn, automatically transfers the sequencing status of the patient to the healthcare network for immediate transfer to the healthcare provider, in the process element 508. Otherwise, the CDS listener device may return to listening for another CDS hook in the healthcare network.

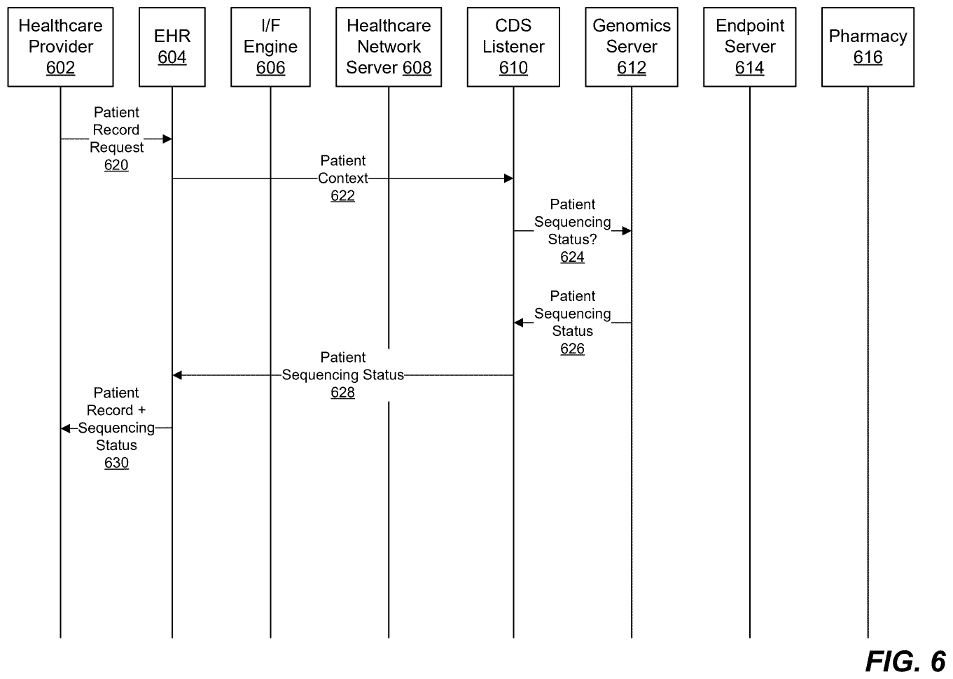

In some embodiments, this may include automatically generating an order for providing genetic test data, or directly providing genetic test data of the patient to the healthcare provider through the healthcare network. For example, if the request from the healthcare provider includes a certain prescription where genetic testing is recommended, and genetic test data for the patient exists or is capable of being rapidly generated via an analytical tool, the genomics server may automatically include a draft order for performing expedited genetic testing. Contrarily, if the genetic test data of the patient is not available, the genomics server may provide a recommendation for standard genetic testing to the listener device, which in turn may automatically provide the recommendation to the healthcare provider through the healthcare network. Other exemplary CDS hook detection and messaging techniques are shown and described below in FIGS. 6-9.

As used herein, a CDS listener device is any device, system, software, or combination thereof operable to detect a CDS hook within a healthcare network.

FIG. 6 is a message diagram of a detected request from a healthcare provider for patient information, in an illustrative embodiment. In this embodiment, a healthcare provider 602 electronically initiates a request 620 for a patient's records from the EHR 604. The EHR 604 provides the patient context 622 with a CDS hook through healthcare network server 608, which is then detected by a CDS listener 610. The CDS listener 610 automatically transfers the patient context to the genomics server 612 to inquire about a sequencing status 624 of the patient. For example, once the genomics server 612 receives a patient identifier from the CDS listener device 610, the genomics server 612 automatically searches its database to determine whether any gene sequencing data for the patient exists. The genomics server 612 then informs the CDS listener 610 of that status 626. And the CDS listener 610 conveys that status 628 to the EHR 604, which may immediately transfer the patient record and the sequencing status 630 to the healthcare provider 602.

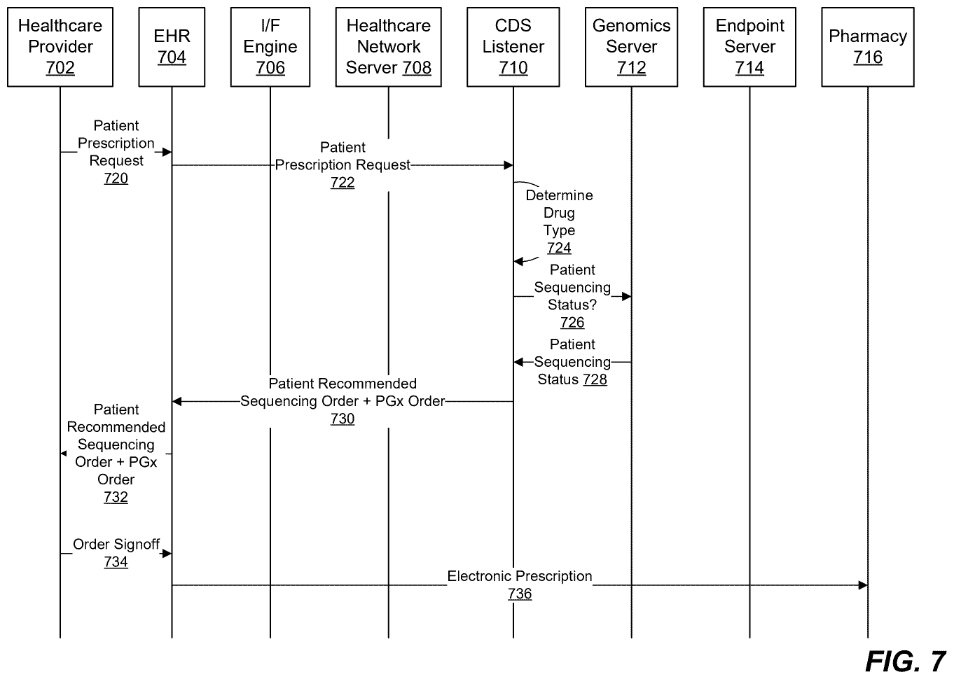

In this illustrative embodiment, FIG. 7 shows a message diagram of a detected prescription order from a healthcare provider and the return of a patient's sequencing status. Here, a healthcare provider 702 electronically requests 720 a prescription for a patient to the EHR 704. The EHR 704 formats the prescription request 722 with the patient information and a CDS hook and then forwards a request 722 through the healthcare network 708. A CDS listener device 710 detects a CDS hook and determines a drug type 724 in the request 722. The CDS listener 710 then contacts the genomics server 712 to determine the sequencing status 726 for the patient. The genomics server 712 returns the sequencing status 728 to the CDS listener 710. If the status 728 indicates that there is no gene sequencing data for the patient, the CDS listener 710 automatically recommends a sequencing order and a PGx order 730 and updates the EHR 704 with such. From there, when the healthcare provider 702 reviews the EHR 704, the healthcare provider 702 can determine that the patient's gene sequencing is needed to fulfill the PGx order 732. The healthcare provider 702 may then provide an order signoff 734 to obtain an electronic prescription 736 from a pharmacy 716. That is, once the healthcare provider 702 approves the patient sequencing and PGx order 732, the EHR 704 automatically provides the electronic prescription 736 to the pharmacy 716 for fulfillment (e.g., mail-order, pick up, etc.), usually after the sequencing and PGx testing is complete. The EHR 704 may automatically initiate the workflow to obtain a biological sample from the patient.

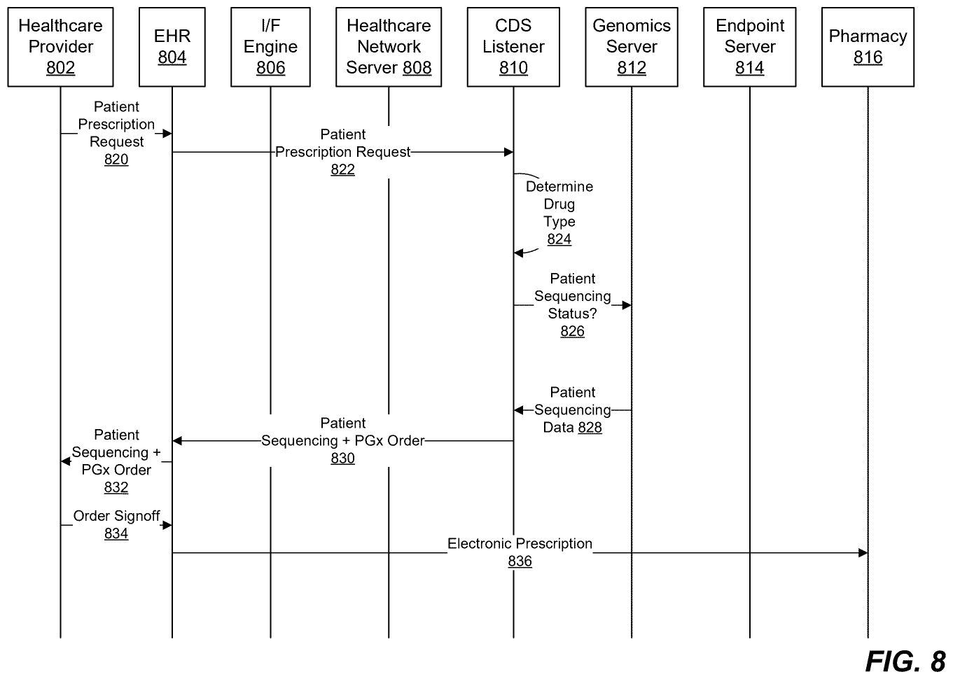

FIG. 8 is a message diagram of a detected prescription order from a healthcare provider and the return of a patient's gene sequencing data, in an illustrative embodiment. Here, the healthcare provider 802 again requests 820 a prescription for a patient by updating the EHR 804. The CDS listener 810 detects the CDS hook pertaining to the patient's prescription request 822 and again determines a drug type 824. The CDS listener 810 contacts the genomics server 812 to determine the patient sequencing status 826. Assuming that the patient has a biological sample that has been sequenced, the genomics server 812 transmits a notification that expedited genetic testing is available for the patient (828). The CDS listener provides the notification 830 to the provider via the EHR 804. Next, the healthcare provider 802 can review 832 the EHR 804 and order expedited genetic testing. Specifically, the healthcare provider 802 can sign off the order 834 in the EHR 804. The EHR 804 then automatically forwards the electronic prescription 836 to the pharmacy 816 for fulfillment.

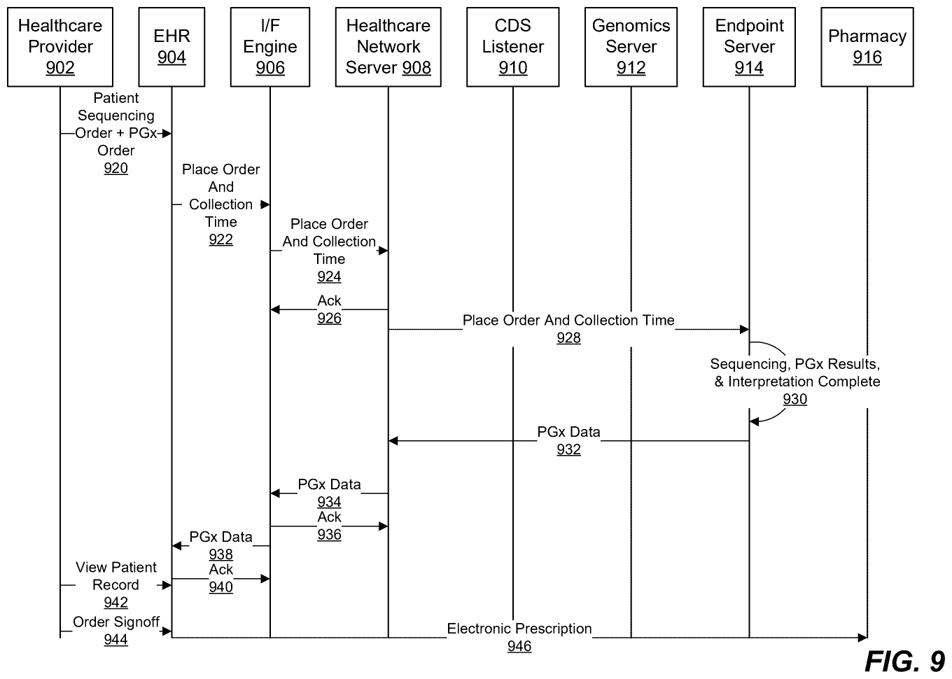

FIG. 9 is a message diagram of a detected prescription order from a healthcare provider and the return of PGx data to the healthcare provider, in an illustrative embodiment. Here, a healthcare provider 902, based on a recommendation for patient sequencing, updates a EHR 904 with a patient sequencing order and PGx order 920. The EHR 904 automatically places the order with a barcode to be used in the patient's biological sample to be sequenced and a collection time 922. An interface engine 906 places the order and collection time 924 within the healthcare network server 908, which in turn acknowledges 926 the order with the interface engine 906. The healthcare network server 908 automatically relays the ordering collection time 928 with the barcode to be used for the patient to an endpoint server 914 of a laboratory, such as the laboratory 190 of FIG. 1. Once the patient's biological sample has been collected, it is transferred to a genomics laboratory, such as the genomics laboratory 120 of FIG. 1, for sequencing. The endpoint server 914 of the laboratory may contact the CDS listener 910 to provide an estimate of the time necessary for sequencing the patient's biological sample. Once the sequencing, PGx results, and interpretation are complete 930, the endpoint server 914 of the laboratory transfers 932 the PGx data to the healthcare network server 908. The healthcare network server 908 then transfers the PGx data 934 to the interface engine 906, which in turn acknowledges receipt 936. The interface engine 906 then updates the EHR 904 with the PGx data 938, which in turn acknowledges receipt 940 to the interface engine 906. From there, the healthcare provider 902 can view 942 the EHR 904 to verify that the patient's PGx data has been obtained such that the healthcare provider can sign off 944 on the prescription order by updating the EHR 904. The EHR 904 then automatically forwards the electronic prescription 946 to the pharmacy 916 for fulfillment.

While the exemplary embodiments herein are shown and described with respect to genetic testing statuses, these embodiments are not intended to be limited to such. Rather, the embodiments herein provide notable improvements over prior techniques because they provide rapid if not instantaneous recommendations to healthcare providers about the patients they serve. Notably, the system actively identifies and reports the availability of genetic testing for specific medications and medical conditions, which a physician might not otherwise be aware of. Furthermore, the dynamic presentation of recommendations based on sequencing status provides a substantial benefit. It saves healthcare providers the time and effort involved in determining a patient's specific status for sequencing, and then customizes an order based on that status. This, in turn, enhances efficiency, and makes the process of sequencing once and querying often a seamless one for healthcare providers. It also saves healthcare providers and patients time, costs, and risks associated with sample collection by avoiding duplicate collections.

Any of the various computing and/or control elements shown in the figures or described herein may be implemented as hardware, as a processor implementing software or firmware, or some combination of these. For example, an element may be implemented as dedicated hardware. Dedicated hardware elements may be referred to as “processors,” “controllers,” or some similar terminology. When provided by a processor, the functions may be provided by a single dedicated processor, by a single shared processor, or by a plurality of individual processors, some of which may be shared. Moreover, explicit use of the term “processor” or “controller” should not be construed to refer exclusively to hardware capable of executing software, and may implicitly include, without limitation, digital signal processor (DSP) hardware, a network processor, application specific integrated circuit (ASIC) or other circuitry, field programmable gate array (FPGA), read only memory (ROM) for storing software, random access memory (RAM), non-volatile storage, logic, or some other physical hardware component or module.

In one embodiment, instructions stored on a computer readable medium direct a computing system of any of the devices and/or servers discussed herein, such as genomics server 220, to perform the various operations disclosed herein. In some embodiments, all or portions of these operations may be implemented in a networked computing environment, such as a cloud computing system. Cloud computing often includes on-demand availability of computer system resources, such as data storage (cloud storage) and computing power, without direct active management by a user. Cloud computing relies on the sharing of resources, and generally includes on-demand self-service, broad network access, resource pooling, rapid elasticity, and measured service.

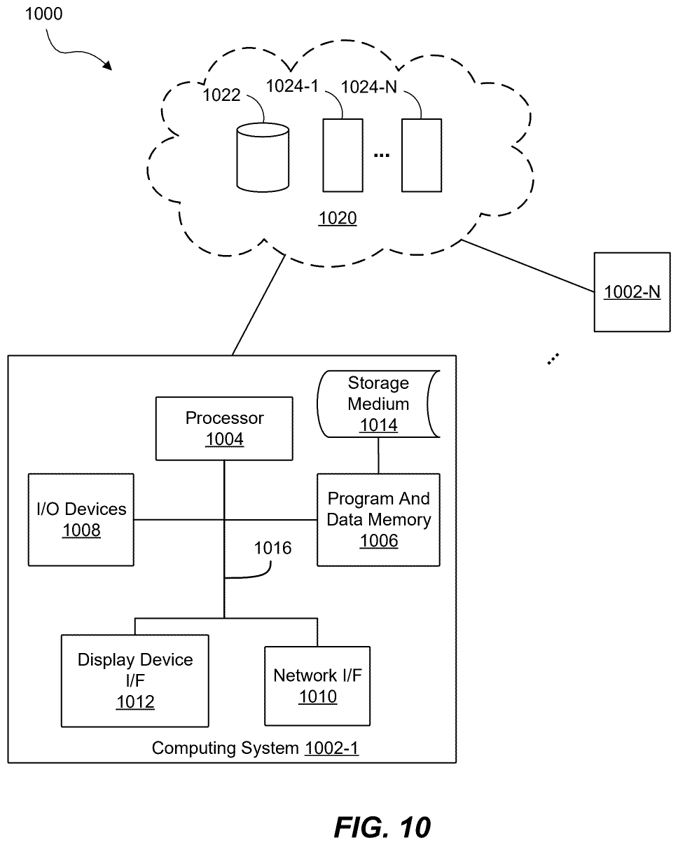

FIG. 10 depicts one illustrative cloud computing system 1000 operable to perform the above operations by executing programmed instructions tangibly embodied on one or more computer readable storage mediums. The cloud computing system 1000 generally includes the use of a network of remote servers hosted on the internet to store, manage, and process data, rather than a local server or a personal computer (e.g., in the computing systems 1002-1, 1002-N). Cloud computing enables users to use infrastructure and applications via the internet, without installing and maintaining them on-premises. In this regard, the cloud computing network 1020 may include virtualized information technology (IT) infrastructure (e.g., servers 1024-1-1024-N, the data storage module 1022, operating system software, networking, and other infrastructure) that is abstracted so that the infrastructure can be pooled and/or divided irrespective of physical hardware boundaries. In some embodiments, the cloud computing network 1020 can provide users with services in the form of building blocks that can be used to create and deploy various types of applications in the cloud on a metered basis.

Various components of the cloud computing system 1000 may be operable to implement the above operations in their entirety or contribute to the operations in part. For example, a computing system 1002-1 may be used to perform analysis of gene sequencing data, and then store that analysis along with the gene sequencing data in a data storage module 1022 (e.g., a database) of a cloud computing network 1020. Various computer servers 1024-1-1024-N of the cloud computing network 1020 may be used to operate on the gene sequencing data and/or transfer the gene sequencing analysis and/or the gene sequencing data to another computing system 1002-N.

Some embodiments disclosed herein may utilize instructions (e.g., code/software) accessible via a computer-readable storage medium for use by various components in the cloud computing system 1000 to implement all or parts of the various operations disclosed hereinabove. Examples of such components include the computing systems 1002-1-1002-N.

Exemplary components of the computing systems 1002-1-1002-N may include at least one processor 1004, a computer readable storage medium 1014, program and data memory 1006, input/output (I/O) devices 1008, a display device interface 1012, and a network interface 1010. For the purposes of this description, the computer readable storage medium 1014 comprises any physical media that is capable of storing a program for use by the computing system 1002. For example, the computer-readable storage medium 1014 may be an electronic, magnetic, optical, electromagnetic, infrared, semiconductor device, or other non-transitory medium. Examples of the computer-readable storage medium 1014 include a solid-state memory, a magnetic tape, a removable computer diskette, a random access memory (RAM), a read-only memory (ROM), a rigid magnetic disk, and an optical disk. Some examples of optical disks include Compact Disk-Read Only Memory (CD-ROM), Compact Disk-Read/Write (CD-R/W), Digital Versatile Disc (DVD), and Blu-Ray Disc.

The processor 1004 is coupled to the program and data memory 1006 through a system bus 1016. The program and data memory 1006 include local memory employed during actual execution of the program code, bulk storage, and/or cache memories that provide temporary storage of at least some program code and/or data in order to reduce the number of times the code and/or data are retrieved from bulk storage (e.g., a hard disk drive, a solid state drive, or the like) during execution.

Input/output or I/O devices 1008 (including but not limited to keyboards, displays, touchscreens, microphones, pointing devices, etc.) may be coupled either directly or through intervening I/O controllers. Network adapter interfaces 1010 may also be integrated with the system to enable the computing system 1002 to become coupled to other computing systems or storage devices through intervening private or public networks. The network adapter interfaces 1010 may be implemented as modems, cable modems, Small Computer System Interface (SCSI) devices, Fibre Channel devices, Ethernet cards, wireless adapters, etc. Display device interface 1012 may be integrated with the system to interface to one or more display devices, such as screens for presentation of data generated by the processor 1004.

What is claimed is:

1. A computer implemented method of a Clinical Decision Support (CDS) listener device, comprising:
detecting, via a CDS hook, a request in a healthcare network from a healthcare provider for a patient;
forwarding the request to a genomics server, the genomics server storing gene sequencing data of a plurality of genetic testing subjects;
determining whether the patient is one of the plurality of genetic testing subjects; and
if the patient is one of the plurality of genetic testing subjects, returning a sequencing status of the patient to the healthcare provider through the healthcare network.

2. The computer implemented method of claim 1, further comprising:
automatically generating a recommendation to provide genetic test data of the patient to the healthcare provider through the healthcare network in response to the detected request.

3. The computer implemented method of claim 1, further comprising:
processing the CDS hook to identify a prescription from the healthcare provider for the patient;
determining that the prescription is for a medicine where genetic testing is recommended;
determining whether genetic test data of the patient exists with the genomics server; and
if the genetic test data of the patient exists with the genomics server, automatically generating a recommendation to provide the genetic test data of the patient to the healthcare provider through the healthcare network,
else, if the genetic test data of the patient does not exist with the genomics server, automatically generating a recommendation for genetic testing of the patient to the healthcare provider through the healthcare network.

4. The computer implemented method of claim 3, further comprising:
processing a request from the healthcare provider through the healthcare network for genetic testing of the patient in response to the recommendation.

5. The computer implemented method of claim 1, further comprising:
detecting another request in the healthcare network from the healthcare provider for another patient, the other request comprising another CDS hook;
processing the other CDS hook to identify a prescription and a genetic test request from the healthcare provider for the other patient;
automatically forwarding the genetic test request to a genetic testing laboratory to perform a genetic test on the other patient; and
when the genetic test is complete, storing data of the genetic test of the other patient with the genomics server, and forwarding the genetic test data of the other patient to the healthcare provider through the healthcare network.

6. The computer implemented method of claim 5, further comprising:
detecting that the healthcare provider has reviewed the genetic test data of the other patient; and
automatically forwarding the prescription to a pharmacy for electronic fulfillment of the prescription.

7. The computer implemented method of claim 1, wherein:
said detecting, forwarding, and returning are implemented via a Session Initiation Protocol.

8. A non-transitory computer readable medium embodying programmed instructions which, when executed by a processor, are operable for performing a method for order processing from a healthcare provider through a healthcare network, the method comprising:
detecting a request in the healthcare network from the healthcare provider for a patient, the request comprising a Clinical Decision Support (CDS) hook;
forwarding the CDS hook to a genomics server, the genomics server storing gene sequencing data of a plurality of genetic testing subjects;
determining whether the patient is one of the plurality of genetic testing subjects; and
if the patient is one of the plurality of genetic testing subjects, returning a sequencing status of the patient to the healthcare provider through the healthcare network.

9. The non-transitory computer readable medium of claim 8, further comprising instructions which, when executed by the processor, are operable for:
automatically generating a recommendation to provide genetic test data of the patient to the healthcare provider through the healthcare network in response to the detected request.

10. The non-transitory computer readable medium of claim 8, further comprising instructions which, when executed by the processor, are operable for:
processing the CDS hook to identify a prescription from the healthcare provider for the patient;
determining that the prescription is for a medicine where genetic testing is recommended;
determining whether genetic test data of the patient exists with the genomics server; and
if the genetic test data of the patient exists with the genomics server, automatically generating a recommendation to provide the genetic test data of the patient to the healthcare provider through the healthcare network,
else, if the genetic test data of the patient does not exist with the genomics server, automatically generating a recommendation for genetic testing of the patient to the healthcare provider through the healthcare network.

11. The non-transitory computer readable medium of claim 10, further comprising instructions which, when executed by the processor, are operable for:
processing a request from the healthcare provider through the healthcare network for genetic testing of the patient in response to the recommendation.

12. The non-transitory computer readable medium of claim 8, further comprising instructions which, when executed by the processor, are operable for:
detecting another request in the healthcare network from the healthcare provider for another patient, the other request comprising another CDS hook;
processing the other CDS hook to identify a prescription and a genetic test request from the healthcare provider for the other patient;
automatically forwarding the genetic test request to a genetic testing laboratory to perform a genetic test on the other patient; and
when the genetic test is complete, storing data of the genetic test of the other patient with the genomics server, and forwarding the genetic test data of the other patient to the healthcare provider through the healthcare network.

13. The non-transitory computer readable medium of claim 12, further comprising instructions which, when executed by the processor, are operable for:
detecting that the healthcare provider has reviewed the genetic test data of the other patient; and
automatically forwarding the prescription to a pharmacy for electronic fulfillment of the prescription.

14. The non-transitory computer readable medium of claim 8, wherein:
said detecting, forwarding, and returning are implemented via a Session Initiation Protocol.

15. A system comprising:
a genomics server operable to store gene sequencing data of a plurality of genetic testing subjects; and
a listening device communicatively coupled to a healthcare network and operable to detect a request in the healthcare network from a healthcare provider for a patient, the request comprising a Clinical Decision Support (CDS) hook,
wherein the listening device is further operable to forward the CDS hook to the genomics server,
wherein the genomics server is further operable to determine whether the patient is one of the plurality of genetic testing subjects, and
if the patient is one of the plurality of genetic testing subjects, the listening device is further operable to return a sequencing status of the patient to the healthcare provider through the healthcare network.

16. The system of claim 15, wherein the listening device is further operable to: automatically generate a recommendation to provide genetic test data of the patient to the healthcare provider through the healthcare network in response to the detected request.

17. The system of claim 15, wherein the listening device is further operable to:
process the CDS hook to identify a prescription from the healthcare provider for the patient;
determine that the prescription is for a medicine where genetic testing is recommended;
determine whether genetic test data of the patient exists with the genomics server; and
if the genetic test data of the patient exists with the genomics server, automatically generate a recommendation to provide the genetic test data of the patient to the healthcare provider through the healthcare network,
else, if the genetic test data of the patient does not exist with the genomics server, automatically generate a recommendation for genetic testing of the patient to the healthcare provider through the healthcare network.

18. The system of claim 17, wherein the listening device is further operable to:
process a request from the healthcare provider through the healthcare network for genetic testing of the patient in response to the recommendation.

19. The system of claim 15, wherein:
the listening device is further operable to detect another request in the healthcare network from the healthcare provider for another patient, the other request comprising another CDS hook, process the other CDS hook to identify a prescription and a genetic test request from the healthcare provider for the other patient, and automatically forward the genetic test request to a genetic testing laboratory to perform a genetic test on the other patient;
when the genetic test is complete, the genomics server is further operable to store data of the genetic test of the other patient with the genomics server; and
the listening device is further operable to forward the genetic test data of the other patient to the healthcare provider through the healthcare network.

20. The system of claim 19, wherein:
the listening device is further operable to detect that the healthcare provider has reviewed the genetic test data of the other patient; and
automatically forward the prescription to a pharmacy for electronic fulfillment of the prescription.

21. The system of claim 15, the listening device is further operable to communicate the healthcare network and the genomics server via a Session Initiation Protocol.
